(19) United States
(12) Patent Application Publication
(10) Pub. No.: US 2009/0198222 A1
(43) Pub. Date: Aug. 6, 2009

(54) Dermal Scar Treatment Equipment

(76) Inventor: Si Hyung Cho, Busan (KR)

Correspondence Address: Hyun Jong Park;TUCHMAN & PARK LLC, 41 White Birch Road, Redding, CT, 06896-2209, US

(21) Appl. No.: 12/055,920

(22) Filed: Mar. 26, 2008

(30) Foreign Application Priority Data
Jan. 31, 2008 (KR) 2008-9947

(51) Int. Cl. A61B 18/20

(52) U.S. Cl. 606/9

(57) ABSTRACT

Provided is a dermal scar treatment equipment. The treatment equipment includes a pad, a plurality of pins, and an insulating layer. The pad electrically connects with a high frequency generator. The pins are protrusively coupled to a front of the pad, are inserted into collagen fibers in the dermis through the epidermis, and propagate a high frequency to the collagen fibers. The insulating layer is coated on an end of each of the pins and isolates the pins from the epidermis for no contact.

### FIELD OF THE INVENTION

The present invention relates to a dermal scar treatment equipment, and more particularly, to a dermal scar treatment equipment for realizing scar treatment by vertically contracting collagen fibers in the dermis with no damage to the epidermis of the scar using a high frequency.

### BACKGROUND OF THE INVENTION

As well-known in the art, skin is composed of the epidermis and the dermis. Below these layers lies a subcutaneous adipose layer. The dermis has a thickness of 1.2 mm to 5 mm.

Collagen fibers in the dermis clump together if skin scars due to a burn and the like. As a result, a burn portion of the skin leaves a rough scar of a rugged shape.

Attempts have been recently made on a diversity of methods for treating such scars. Among the methods, the most representative technology can be a scar treatment equipment using a fractional laser, for example.

The fractional laser technology is based on the principle of making a fine hole in skin using a laser, infiltrating a stimulus of heat into the dermis, and promoting collagen formation for reproduction. It has been reported that the fractional laser technology exhibits a remarkable effect on a scar caused by pimple, pore reduction, wrinkles, brightening, lifting or the like in addition to medical scar treatment.

However, the fractional laser technology needs a long time to take effect on a hypertropic scar and also, causes side effects of increasing a rate of generation of pigmentation after erythema or inflammation if medical treatment is performed using an increased energy.

### SUMMARY OF THE INVENTION

An aspect of exemplary embodiments of the present invention is to address at least the problems and/or disadvantages and to provide at least the advantages described below. Accordingly, an aspect of exemplary embodiments of the present invention is to provide a dermal scar treatment equipment for realizing scar treatment by inserting a plurality of pins into collagen fibers clumping together in the dermis of a scar portion and then propagating a high frequency to the pin-inserted collagen fibers, thus vertically contracting the collagen fibers, reducing a thickness of the dermis, and fragmentizing the collagen fibers.

Another aspect of exemplary embodiments of the present invention is to provide a dermal scar treatment equipment for preventing a high frequency heat from burning or damaging the epidermis overlying the dermis during a medical treatment process, by forming an insulating layer around pin ends.

According to one aspect of exemplary embodiments of the present invention, there is provided a dermal scar treatment equipment. The treatment equipment includes a pad, a plurality of pins, and an insulating layer. The pad electrically connects with a high frequency generator. The pins are protrusively coupled to a front of the pad, are inserted into collagen fibers in the dermis through the epidermis as being kept at regular intervals, and propagate a high frequency generated in the high frequency generator to the collagen fibers. The insulating layer is coated on an end of each of the pins and isolates the pins from the epidermis for no contact.

### BRIEF DESCRIPTION OF THE DRAWINGS

The above and other objects, features and advantages of the present invention will become more apparent from the following detailed description when taken in conjunction with the accompanying drawings in which:

FIG. 1 is a diagram illustrating the entire construction of a dermal scar treatment device according to the present invention;

FIG. 2 is a three-dimensional diagram illustrating a dermal scar treatment equipment taken from, shown in FIG. 1;

FIG. 3 is a diagram illustrating a use state of the dermal scar treatment equipment shown in FIG. 2; and

FIGS. 4A to 4H are snapshots showing states before and after treatment of patient's scar in clinical trials using a dermal scar treatment equipment according to the present invention.

Throughout the drawings, the same drawing reference numerals will be understood to refer to the same elements, features and structures.

### DETAILED DESCRIPTION OF THE INVENTION

Exemplary embodiments of the present invention will now be described in detail with reference to the annexed drawings. In the following description, a detailed description of known functions and configurations incorporated herein has been omitted for conciseness.

A dermal scar treatment equipment proposed in the present invention is used for treating a hypertropic scar. The dermal scar treatment equipment is characterized in that it realizes scar treatment by inserting a plurality of pins electrically connecting to a pad into collagen fibers that clump together in the dermis of a scar portion and then applying a high frequency to the pins to fragmentize the clumping collagen fibers by the high frequency. Particularly, the collagen fibers are vertically contracted during a high frequency propagation process, thereby consequently reducing a thickness of the scar portion.

In particular, the present invention is configured to limitedly propagate a high frequency not to the entire collagen fibers in the dermis but only to collagen fibers of locations where a plurality of pins are inserted, to minimize the destruction of collagen fibers caused by a high frequency heat.

Also, in a process of propagating high frequency energy to the dermis using a partial high-frequency propagation scheme, collagen fibers of locations where the pins are inserted are contracted because of a high frequency heat. At this time, the collagen fibers draw adjacent different collagen fibers. This drawing effect leads to fragmentizing the collagen fibers clumping together in the dermis and flattening a rough scar portion of a rugged shape like a non-scarred peripheral skin. Thus, more effective scar treatment can be achieved.

In the present invention, conductivity is not realized throughout all pins propagating a high frequency but insulating layers are limitedly formed only at ends of the pins. This allows the insulating layers formed at the pin ends to get in contact with the epidermis overlying the dermis when the pins are inserted into the dermis. Thus, the insulating layer prevents the propagation of a high frequency heat to the epidermis, thereby being able to prevent a damage of the epidermis during treatment.

### Exemplary Embodiment 1

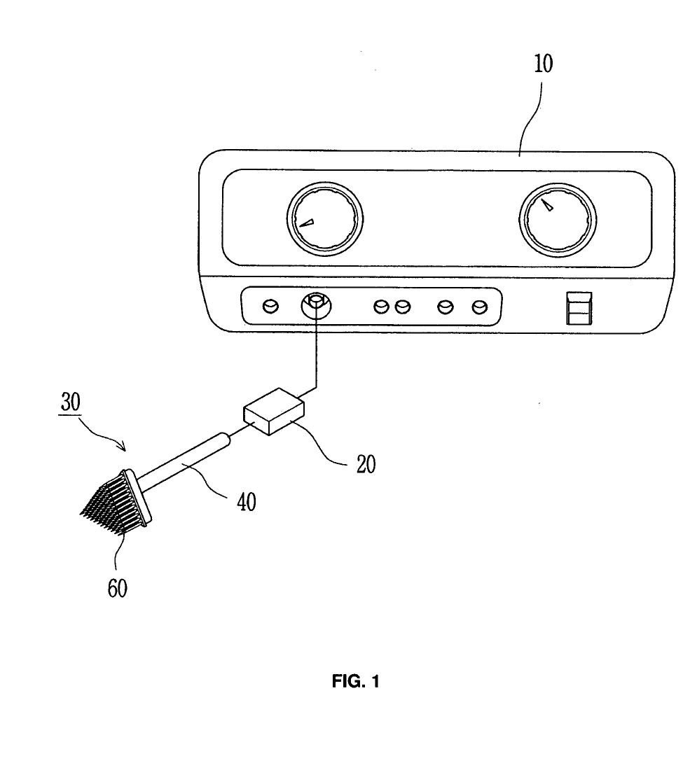

FIG. 1 is a diagram illustrating the entire construction of a dermal scar treatment device according to the present invention.

As shown in FIG. 1, the dermal scar treatment device includes a high frequency generator 10, a connector 20, and a dermal scar treatment equipment 30. The connector 20 connects to the high frequency generator 10. The dermal scar treatment equipment 30 detachably electrically connects to the connector 20 and receives a high frequency generated in the high frequency generator 10.

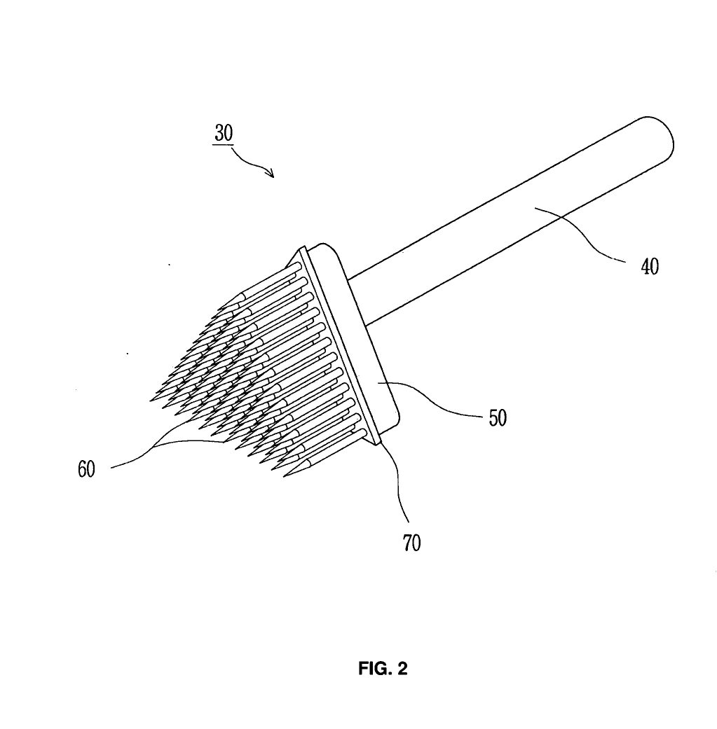

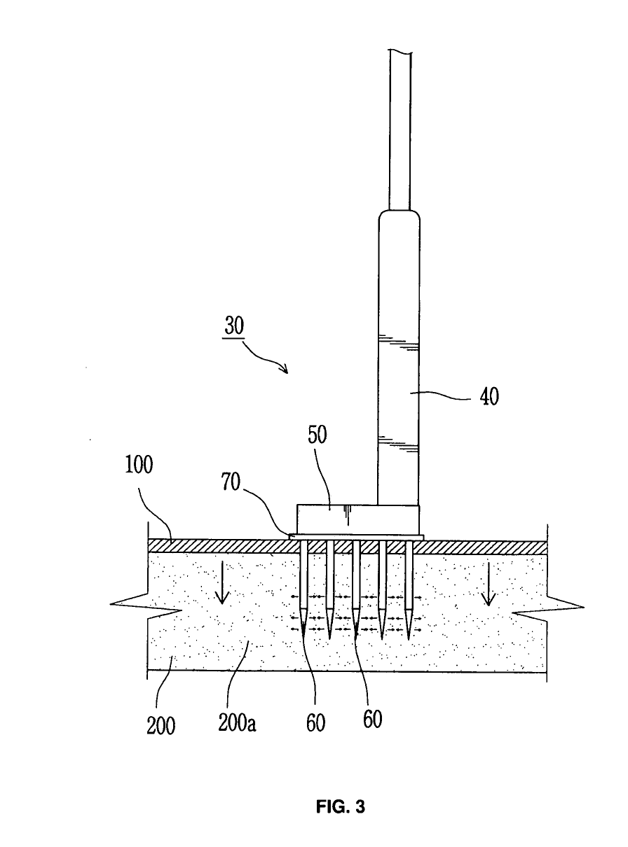

FIG. 2 is a three-dimensional diagram illustrating a detailed construction of a dermal scar treatment equipment according to an exemplary embodiment of the present invention. FIG. 3 is a diagram illustrating a use state of the dermal scar treatment equipment shown in FIG. 2.

As shown in FIGS. 2 and 3, the dermal scar treatment equipment 30 includes a rod 40, a flat pad 50, and a plurality of pins 60. The rod 40 is electrically detachably coupled to the connector 20. The flat pad 50 is coupled to a front end of the rod 40. The pins 60 are electrically coupled to a front surface of the pad 50.

As being inserted into collagen fibers 200a clumping together in the dermis 200 of a hypertropic scar that is a scar portion, the pins 60 receive a high frequency generated in the high frequency generator 10 via the rod 40 and the pad 50 and propagate the received high frequency to the collagen fibers 200a. A phenomenon of vertically contracting the collagen fibers 200a receiving the high frequency through the pins 60 occurs. This phenomenon leads to a reduction of the entire thickness of the dermis 200 suffering the hypertropic scar. Consequently, an effect of treating the hypertropic scar can be realized.

In the present invention, a plurality of the pins 60 are coupled and formed on the pad 50 as being kept at regular intervals. Forming the pins 60 in plurality is to fragmentize the collagen fibers 200a in the dermis while minimizing the destruction of the collagen fibers 200a.

That is, as shown in FIG. 3, if the pins 60 receive a high frequency after being inserted into the collagen fibers 200a in the dermis, a high frequency heat thus generated is propagated only to collagen fibers 200a of a portion into which the pins 60 are inserted. Through this process, the collagen fibers 200a are contracted and thus draw adjacent different collagen fibers 200a into which the pins 60 are not inserted. Consequently, the collagen fibers 200a clumping together are fragmentized. Also, the destruction of the collagen fibers 200a caused by the high frequency heat can be minimized because the high frequency heat is limitedly propagated only to the collagen fibers 200a into which the pins 60 are inserted.

The ends of the pins 60 are coated with insulating layers 70. The insulating layers 70 isolate the pin ends from the epidermis 100 for no contact between them when the pins 60 are inserted into collagen fibers 200a of the dermis 200 after passing through the epidermis 100 of the hypertropic scar. Thus, the insulating layers 70 prevent a burn or damage of the epidermis 100 caused by the high frequency heat propagated from the pin 60.

An experiment example of a surgical operation method for treating a hypertropic scar using a dermal scar treatment equipment according to the present invention is described below.

### Experiment Example 1

Objects for treatment were 20 patients suffering a hypertropic burning scar who had visited KIM YANG JE dermatology and BUSAN GOWOON SESANG dermatology that are located at Busan in Korea. The experiment result was obtained from the follow-up observation for one year after surgical operation under patients' approval for clinical trials.

Surgical operation method for treatment was performed by executing local lidocaine anesthesia for an operation portion having a hypertropic scar, then inserting the pins 60 of the dermal scar treatment equipment 30 into the dermis 200 through the epidermis 100 of the operation portion with the pins 60 standing at right angles, and then applying a high frequency to the pins 60 inserted into the dermis 200 with adhering a front of the pad 50 to the epidermis 100.

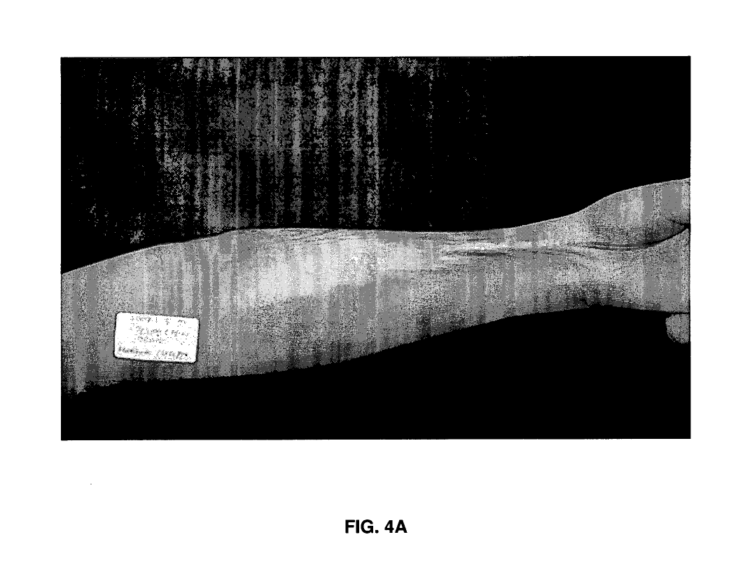

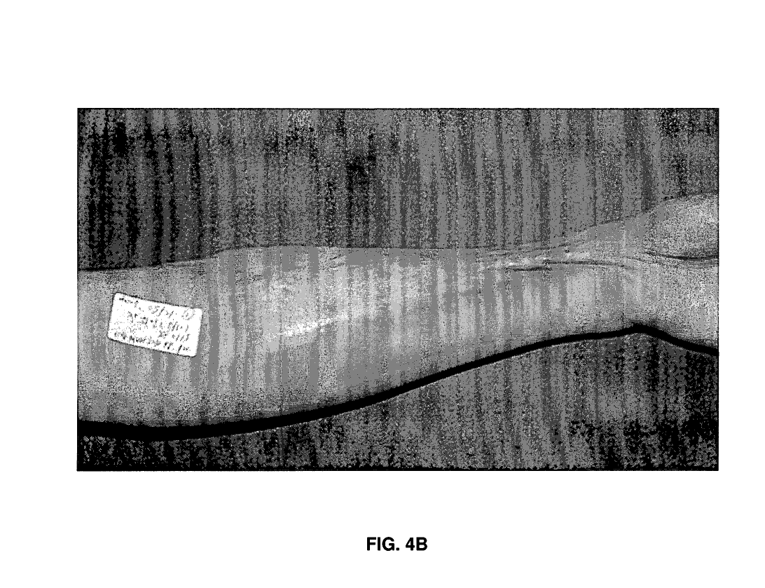

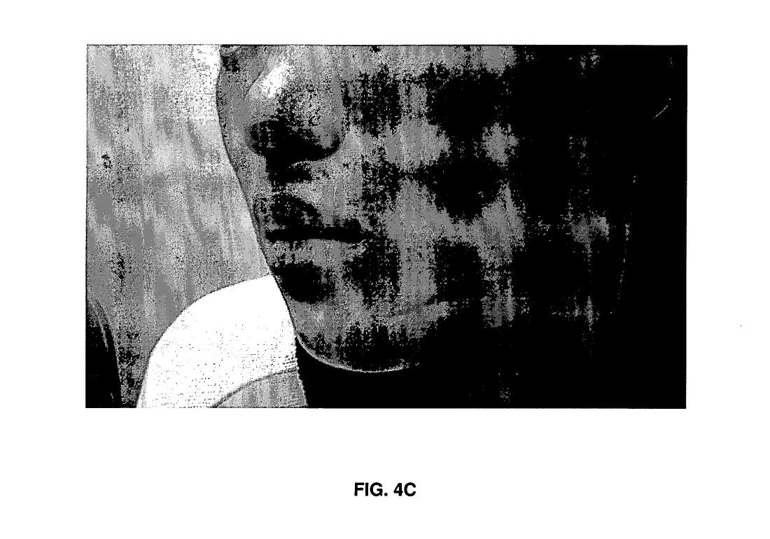

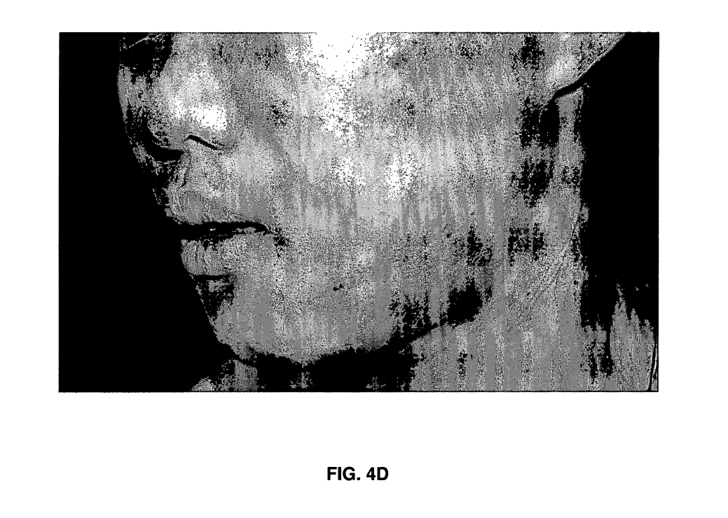

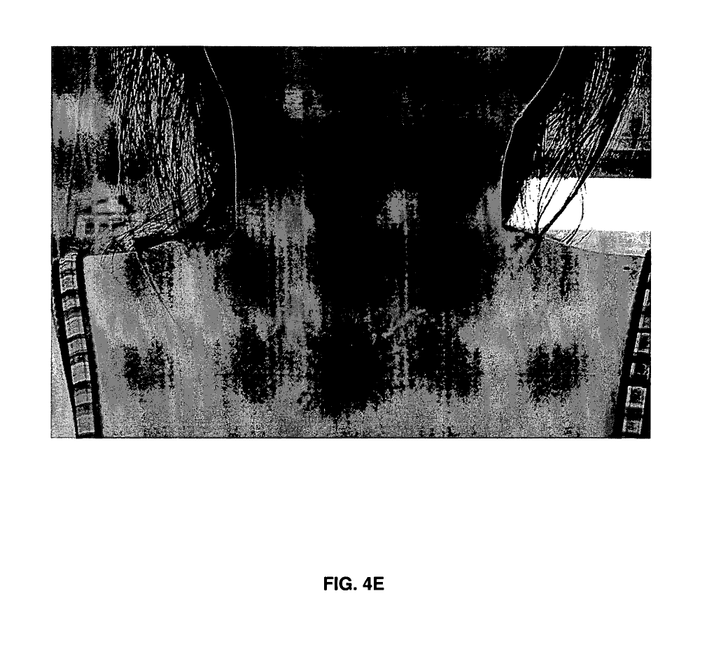

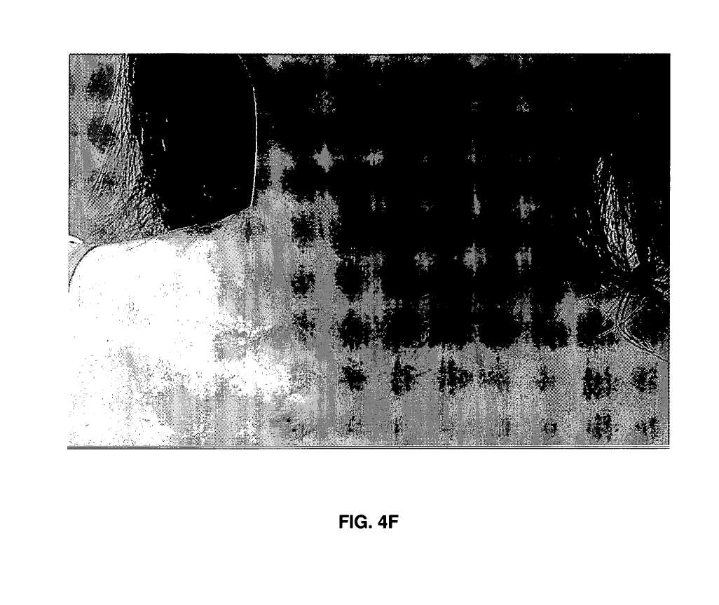

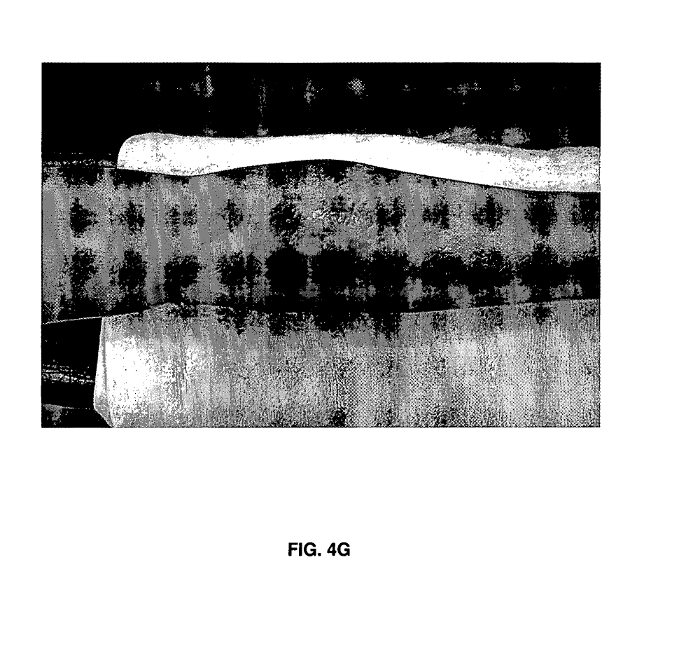

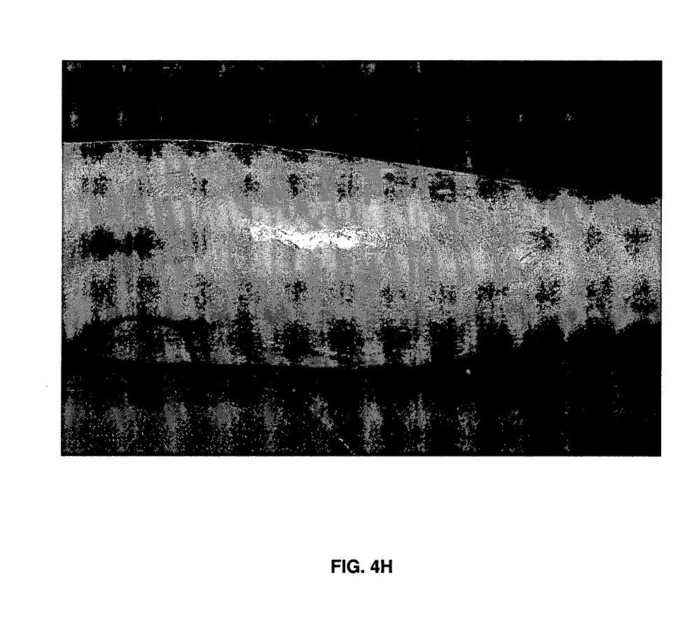

As the experiment result of follow-up observation for one year along with a survey of object patients, it was reported that there was no erythema or Post Inflammatory Hyperpigmentation (PIH) except edema of a surgical operation portion generated for down time of about one to two days as in snapshots of FIGS. 4A to 4H taken before and after treatment of a hypertropic scar on a patient's wrist, neck, face. It was reported that most of the object patients suffered a reduction of a thickness of lesion, flattening and smoothening of the entire of the surgical operation portion, and a reduction of erythema.

The dermal scar treatment equipment of the present invention achieves effects as follows.

The present invention is configured to insert a plurality of pins into collagen fibers, which clump together in the dermis of a scar portion, and then apply a high frequency to the pins, thereby being able to vertically contract, by a high frequency heat, the collagen fibers of a portion into which the pins are inserted, reduce a thickness of the dermis, and fragmentize the collagen fibers during a contraction process. Thus, effective scar treatment can be achieved.

Also, the present invention has an effect of being capable of preventing the epidermis overlying the dermis from being burned or damaged because of a high frequency heat during a treatment process by forming insulating layers at ends of pins to avoid a direct contact between the pins and the dermis.

While the invention has been shown and described with reference to a certain preferred embodiment thereof, it will be understood by those skilled in the art that various changes in form and details may be made therein without departing from the spirit and scope of the invention as defined by the appended claims.

What is claimed is:

1. A dermal scar treatment equipment comprising:
a pad electrically connecting with a high frequency generator;
a plurality of pins protrusively coupled to a front of the pad, inserted into collagen fibers in the dermis through the epidermis as being kept at regular intervals, and propagating a high frequency generated in the high frequency generator to the collagen fibers; and
an insulating layer coated on an end of each of the pins and isolating the pins from the epidermis for no contact,
wherein the equipment partially propagates a high frequency to the collagen fibers in the dermis underlying the epidermis and vertically contracts the collagen fibers to treat a scar.
